(19) United States
(12) Patent Application Publication
(10) Pub. No.: US 2005/0069175 A1
(43) Pub. Date: Mar. 31, 2005

(54) Image/optical analysis of citrus pulp

(75) Inventors: Juliana Parente, Bradenton, FL (US); Jennifer Dicicco, St. Petersburg, FL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(73) Assignee: Tropicana Products, Inc.

(21) Appl. No.: 10/673,732

(22) Filed: Sep. 29, 2003

(51) Int. Cl. G06K 9/00

(52) U.S. Cl. 382/110

(57) ABSTRACT

A method for incorporating instrumentation in determining sensory quality of citrus pulp in citrus juice by measuring, using image-based measurements, one or more parameters of the pulp in a sample of the citrus juice, linking the parameter measurements to sensory evaluations, and making a sensory determination of the quality of the pulp content in the juice, which is linked to consumers acceptance of the amount of pulp in the juice is disclosed.

### FIELD OF THE INVENTION

The present invention is directed to a method for image-based measurement of particles and pulp parameters to determine citrus pulp size and sensory quality in citrus juice.

### BACKGROUND OF THE INVENTION

Many consumers prefer pulp in their citrus juice. It has been discovered that the presence of larger sizes of citrus pulp within citrus juices is an indicator of positive sensory quality for consumers who like pulp in citrus juices.

Currently, during the commercial production of pulpy citrus juices, a series of screens of various sieve sizes are used by an operator to sift the juice and pulp within the juice. The screens are then weighed to quantify the amount of pulp on each screen. For example, the screens will be weighed to determine how much or how many grams of small sized pieces, medium sized pieces and large pieces are on the various screens, based on the sieve size of the respective screens.

The current method has several disadvantages. For example, the results of the current method are very operator dependent, and accordingly, the results usually are not repeatable by different operators. Further, the current method is a lengthy procedure which has high variation in test results from test to test, even if done by the same operator.

The current method also does not accurately determine the pulp quality of each of the measured size distribution quantities. As a result, a separate sensory evaluation of the juice is often necessary to obtain information with regard to whether the tested juice has acceptable pulp and pulp levels to meet the expectations of consumers who like pulp in their juice. Such sensory evaluation is a very time-consuming method that requires a panel of experts in pulp attributes to evaluate the perceived quality of pulp visually and through placing the pulp within the mouth of each tester.

Accordingly, it is an object of the present invention to provide a method to accurately and consistently measure one or more parameters of pulp for citrus juice and link such parameters to sensory evaluations to arrive at a conclusion as to whether the pulp in the tested juice is acceptable to consumers.

### SUMMARY OF THE INVENTION

The present invention is directed to a method for incorporating instrumentation in determining sensory quality of citrus pulp in citrus juice by measuring, using image-based measurements, one or more parameters of the pulp in a sample of the citrus juice, linking the parameter measurements to sensory evaluations, and making a sensory determination of the quality of the pulp content in the juice, which is linked to consumers acceptance of the amount of pulp in the juice.

In a preferred embodiment of the present invention, the method comprises measuring one or more parameters of pulp in a sample of juice using image based measuring; and comparing the measured parameter(s) to known sensory evaluations to determine a sensory quality of pulp in the citrus juice and correlating the sensory quality of pulp to known consumer ratings to determine whether the citrus juice has an acceptable amount of the type of pulp desired by consumers.

In a further embodiment of the present invention, the measured parameter(s) of pulp is compared to an established relationship between the parameter(s) and sensory perceived quantity in the mouth to determine a sensory perceived quantity in the mouth for the measured parameter(s). In yet a further embodiment, the sensory perceived quantity in the mouth for the measured parameter(s) is compared to an established relationship between sensory perceived quantity in the mouth and consumer ratings of pulp amount to predict consumer acceptability for the sensory perceived quantity in the mouth for the measured parameter(s).

Preferably the present invention is directed to use in the evaluation and monitoring of pulp quality in citrus juice, such as orange, grapefruit, tangerine, lemon and combinations or blends thereof. More preferably, the juice is orange or grapefruit juice.

### BRIEF DESCRIPTION OF THE DRAWINGS

FIG. 1 is an illustration of a bench top instrument for measuring particle size using computerized image analysis, in accordance with a method of the present invention.

FIG. 2 shows an example of a computer image of a sample.

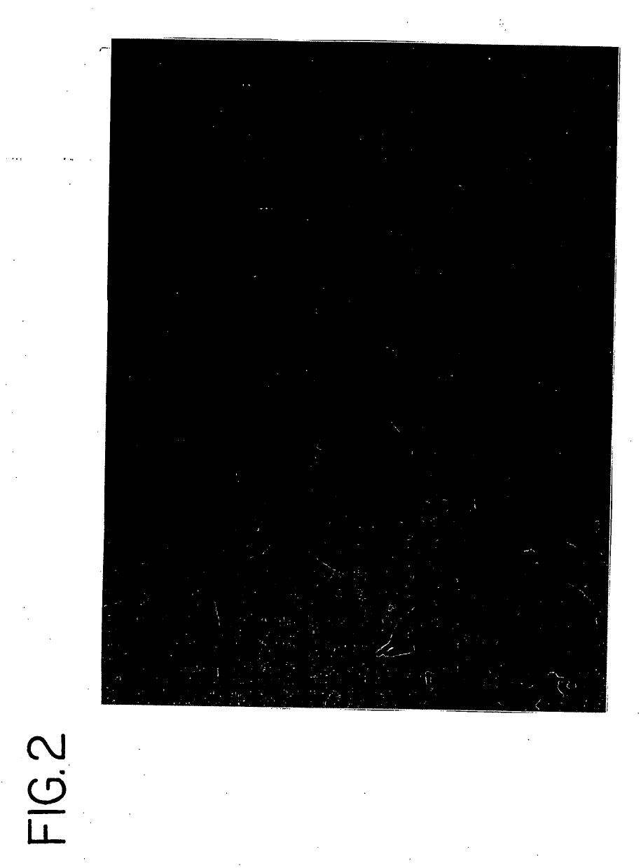

FIG. 3 is a graphical representation of an example of fibre length of pulp versus sensory texture ratings of pulp quantity based on multiple samples of pulp.

FIG. 4 shows a graphical representation of an established relationship between sensory pulp quantity scores and consumer ratings of “just right pulp.”

### DETAILED DESCRIPTION OF THE PRESENTLY PREFERRED EMBODIMENTS

The present invention is directed to a method for image-based measurement of particles to determine the parameters, such as for example size, and also quality of citrus pulp in citrus juice. In accordance with the method, such image-based measurement can be used to quantify the amount of large pulp cells or sacs, to predict sensory quality of a pulp sample, and to track pulp quality over time or other variables. Pulp cells are fibrous sacs holding juice or erupted sacs floating in the juice. The information from the measurements can be used to establish quality control measures and criteria for use on commercial products to consistently produce a juice with the pulp, sensory qualities and mouth feel which had been selected and characterized as highly desirable targets.

In one embodiment of the method of the present invention, a sample of pulp, preferably citrus pulp, is taken from a juice sample in question, and one or more parameters of the pulp in the juice are measured. Preferably, the parameters are measured using an image-based measurement. The parameter measurements are then outputted to the operator, preferably in a spreadsheet form. For example, the maximum and minimum of a parameter, such as for example pulp length, in the sample can be determined and stored as needed. Statistical analysis preferably then are conducted on such parameter measurements.

As an alternative to a single measurement, more than one measurement can be taken, and a range of measurement or average measurements can be calculated, and the range or average subjected to the statistical analysis. The resulting statistical analysis predicts a sensory rating score. The sensory rating score represents, for example, the percentage of large pulp in the sample. The sensory rating score is then compared to previously compiled research regarding such scores and the relationship between such scores and consumer evaluations of pulp in citrus juice. Based on such results, a determination can then be made as to whether this juice has the right size of pulp to be acceptable to consumers who like pulp in their juice.

For example, once a parameter measurement has been outputted to the operator, the parameter measurement can be compared to previously compiled and established data between parameter measurements and sensory measurements of perceived pulp quantity in the mouth to determine a sensory perceived pulp quantity in the mouth for the outputted parameter measurement. Sensory perceived pulp quantity in the mouth has been found to relate to the size and integrity of the pulp when measured at a constant density. Data also has been developed that establishes a relationship between sensory measurements of pulp quantity in the mouth and consumer rating of pulp amount. Using the method of the present invention, the results of the evaluation of sensory perceived pulp quantity in the mouth determined above can be compared to the established data to predict consumer ratings of just right pulp amount. The resulting predicted consumer rating will allow the operator to determine the acceptability of the pulp in the sampled juice.

In another illustration, results of the analysis of a sample might show that the sample has a measured parameter of “x mm” of length. This measured parameter will then be compared to the previously compiled existing research showing the relationship of percent of pulp and sensory measurement of perceived pulp quantity in the mouth which results in a “y” sensory measurement of perceived pulp quantity. This resulting “y” sensory measurement of pulp quantity in the mouth is then compared to previously compiled research showing the relationship between sensory mouth measurement of pulp quantity in the mouth and consumer ratings of pulp amount. As a result, the operator can reach a conclusion whether the tested juice having “x mm” of length is satisfactory to consumers who like pulp in their juice.

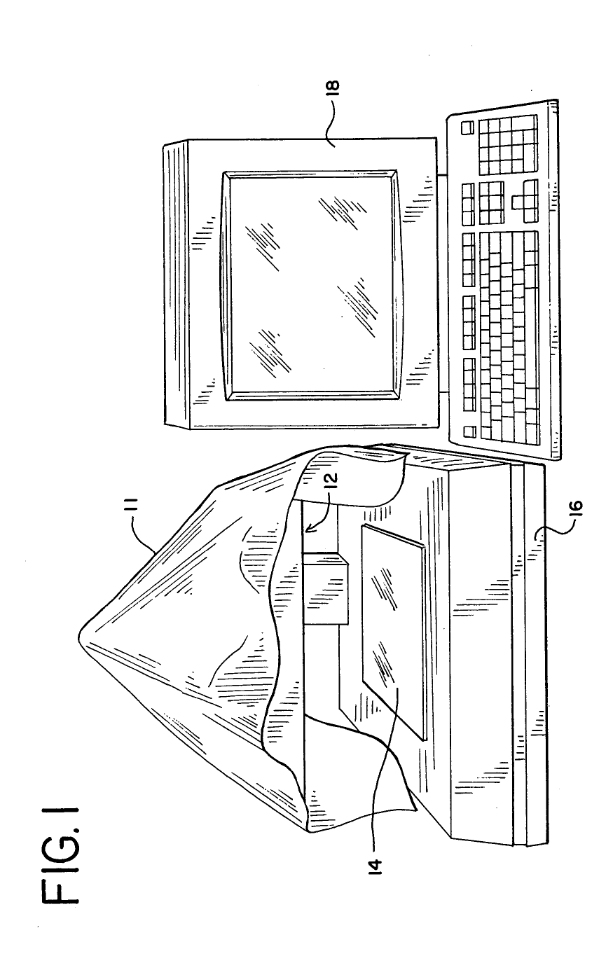

In one preferred embodiment of the method of the present invention, a particle image analyzer is used to measure parameters, such as, for example determining the size distribution of citrus pulp sacs in a given sample. For example, a bench top instrument that measures particle size using computerized image analysis can be used. FIG. 1 shows an example of such a device. The device 10 of FIG. 1 includes a camera stand 11 with covered camera 12, a sample plate 14, a sample base with light source 16 and a computer (with monitor) 18. The camera 12 preferably links directly to spreadsheet software, such as for example Microsoft Excel spreadsheet software, within computer 18.

In the method of the present invention using device 10, a sample of orange citrus pulp is placed on sample plate 14. Light source 16 is turned on, and camera 12 captures the image. Computer 18 then records the image, and measurements of various parameters of the particles in the sample are then determined. Such measurements can be made by, for example, pixel counting. Types of measurements include length, area, perimeter, fibre width, fibre length, equivalent diameter, circularity, axial ratio and mean grey level. This particle data from camera 12 is then sent to computer 18 which typically records the data, converts it and enters the data in a spreadsheet, such as an Excel spreadsheet. The pixel data can be converted, for example, into millimeters of length. The spread sheet and data are then displayed for the user. The data generated in the spreadsheet can be used in independent statistical analysis to evaluate pulp size distribution and sensory quality, as explained above.

One example of an apparatus which can be used in the above embodiment of the present invention is a 900-220 Optomax Sorcerer Image/Particle Size analyzer. Preferably, this analyzer includes image analysis hardware, particle counting/sizing/shape software with filters, image save and retrieve/sharpen functions, auto detect/local area matrix detection, a monochrome CCD camera (768×576 pixels) with electronic shutter, and a computer. This apparatus is from Optomax of Hollis, N.H. The Optomax particle characterization instrument performs image analysis by pixel counting. Preferably, the Optomax measures area, perimeter, longest dimension, fibre width, fibre length, equivalent diameter, circularity, axial ratio and mean grey level.

In tests run using the Optomax analyzer, measurements of area, longest dimension and perimeter of pulp were found to be fairly consistent from test to test. It was also found that the evaluation date and length of down time of the machine did not have a significant effect on the results.

The following examples are meant to illustrate but not limit the present invention.

### EXAMPLE 1

Table 1 shows an example of the correlation between measurements of pulp parameters made using an Optomax, in the manner described above, and the sensory trained descriptive panel measurements of pulp sensory attributes.

TABLE 1
Correlation Between Sensory and Optomax Measures
Phase II, Hamlin, Valencia 2002 and 2003, Blended and Late 2002 Samples

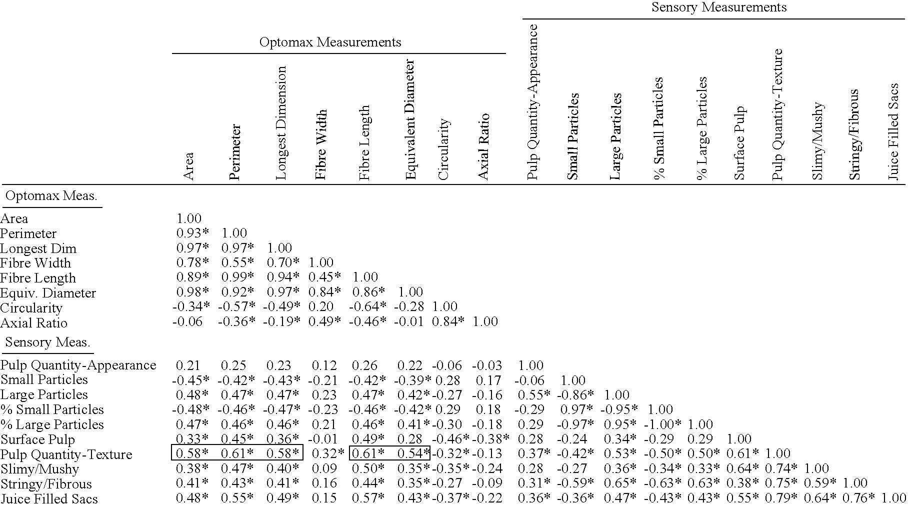

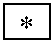

= statistically significant correlations (p < 0.05)

The boxes in Table 1 highlight the Optomax measurements that are most highly correlated with the sensory texture measurement of pulp quantity. The Optomax measurements of perimeter and fibre length had the highest correlation with sensory pulp quantity-texture (r=0.61).

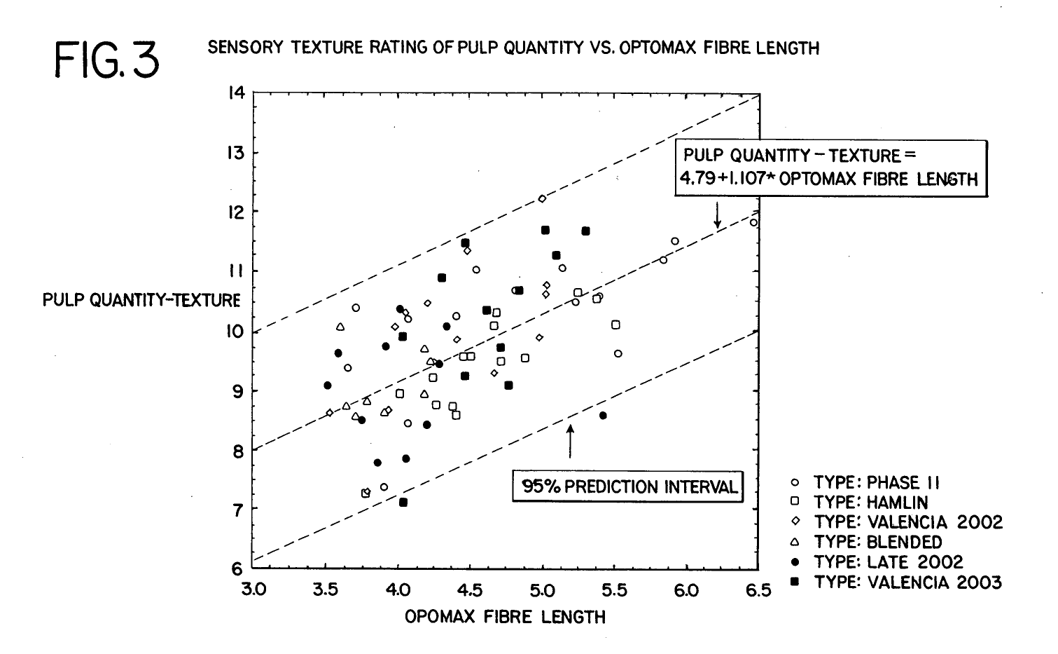

FIG. 3 shows the relationship between measurements of fibre length taken in accordance with the method of the present invention using an Optomax and sensory texture ratings of pulp quantity for 77 samples of pulp from 6 orange juice sources. As there is a statistically significant linear relationship between fibre length and sensory pulp quantity-texture, this relationship can be used in accordance with the method of the present invention for comparing a measured parameter of fibre length from a pulp sample to the established relationship to determine the sensory pulp quantity of the measured parameter from the pulp sample.

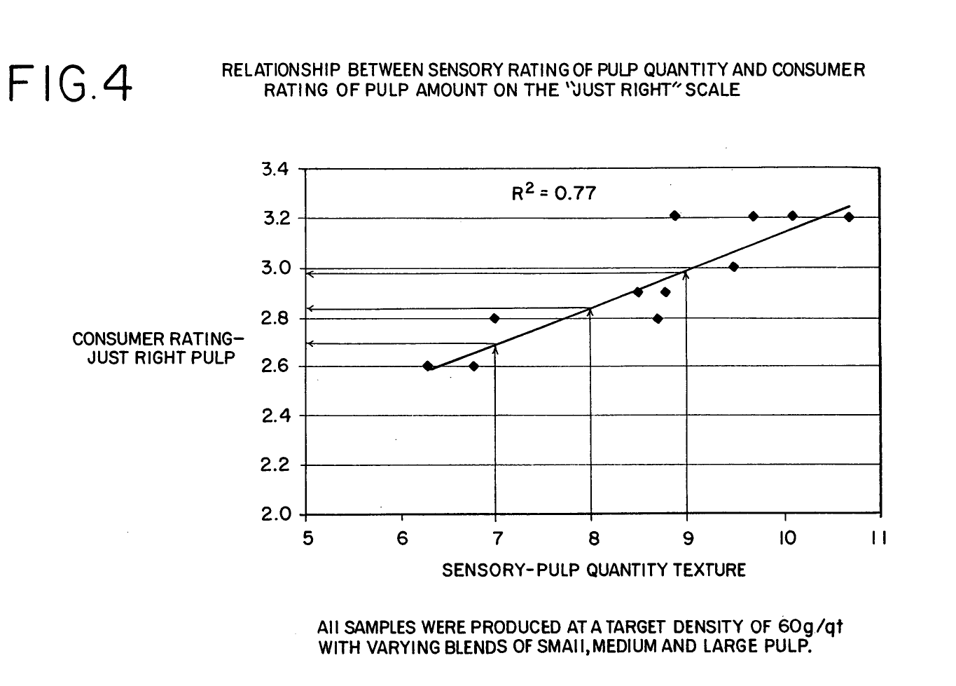

FIG. 4 shows an established relationship between sensory pulp quantity scores and consumer ratings of “just right pulp.” Sensory pulp quantity relates to the size when measured at constant concentration. It has previously been determined that for the consumer ratings:
3.0 equals just right pulp;
<3.0 equals too little pulp; and
>3.0 equals too much pulp.

Hence, as shown in FIG. 4, a sensory pulp quantity texture rating of 9 is closest to a consumer rating of 3.0 (i.e. “just right pulp”), though a sensory pulp quantity texture rating of 8 has been found to be acceptable to consumers. Further, a sensory pulp quantity texture rating of 7 has been found to be a minimum acceptance rating for consumers. However, many consumers who like pulp in their juice perceive this to be too little pulp.

This relationship between sensory pulp quantity scores and consumer ratings of just right pulp can be used in accordance with the method of the present invention for comparing the calculated sensory pulp quantity of the measured parameter from a pulp sample with this established relationship to determine the consumer rating, on the “just right” scale, for the measured parameter of the pulp sample. For example, if the sample has a sensory pulp quantity rating of between 8-9, then the juice from which the sample was taken should be satisfactory to those who like pulp in their juice.

### EXAMPLE 2

In this example, the method of the present invention was tested against the current screening method (a.k.a. the size distribution method) to determine which is more accurate.

Samples were produced from stored Valencia orange pulp. For the present invention, an Optomax analyzer was used. For each test done, correlations were derived between the known sample compositions and the measured values from the method of the present invention and the current screening method.

Results

For % of small particles of pulp, the present invention had a correlation of 0.820 while the current screening method had a correlation of 0.726. This indicates that the present invention had better correlation than the current screening method.

For % of medium particles of pulp, the present invention had a correlation of 0.628 while the current screening method had a poor correlation of 0.296.

For % of large particles of pulp, the present invention had a correlation of 0.793 while the current screening method had a poor correlation of 0.387.

The results show that the method of the present invention is far better at distinguishing between various sized samples than the current screening method as it relates to the size of the samples.

When testing the method of the present invention for determining sensory % of large pulp, there was a correlation of 0.92 between the predicted results using the method of the present invention and the known actual % of large pulp in the sample. Sensory % of large pulp has been shown to be important information as there is a strong correlation between the sensory % of large pulp and consumer acceptance.

In addition to the examples given herein, other technologies can be used to practice the method of the present invention. For example, Time of Transmition (TOT) using a He-Ne laser beam or other known light sources can be used within the scope of the invention. Different sample handling methods can also be used within the scope of the invention such as for example, static or moving samples.

It will be understood that the embodiments and examples of the present invention, which have been described, are illustrative of some of the applications of the principles of the present invention. Numerous modifications may be made by those skilled in the art without departing from the spirit and scope of the invention.

1. A method for determining sensory quality of pulp in citrus juice comprising:
measuring a parameter of pulp in a sample of juice using image based measuring; and
comparing said measured parameter to known sensory evaluations to determine a sensory quality of pulp in the citrus juice and correlating said sensory quality of pulp to known consumer ratings to determine whether the citrus juice has the type of pulp desired by consumers.

2. The method of claim 1 wherein more than one parameter of said pulp is measured and compared to known sensory evaluations.

3. The method of claim 1 wherein said parameter is selected from the group consisting of length, area, perimeter, fibre width, fibre length, equivalent diameter, and circularity.

4. The method of claim 1 wherein said citrus juice is selected from the group consisting of orange, grapefruit, tangerine, lemon and blends thereof.

5. The method of claim 1 wherein said image based measuring is done using computerized image analysis.

6. The method of claim 1 wherein said parameter measurements are provided to the user in a spread sheet.

7. The method of claim 1 wherein said measured parameter is compared to an established relationship between said parameter and sensory perceived quantity in the mouth to predict a sensory perceived quantity in the mouth for said measured parameter.

8. The method of claim 7 wherein said sensory perceived quantity in the mouth for said measured parameter is compared to an established relationship between sensory perceived quantity in the mouth and consumer rating of pulp amount to determine a consumer rating for said sensory perceived quantity in the mouth for said measured parameter.

9. The method of claim 1 wherein said image based measuring is done using a particle image analyzer.

10. A method for determining sensory quality in citrus generating pulp comprising:
generating more than one measurement of a parameter of pulp in said sample of pulp using image based measuring;
determining a range of said measured parameters; and
comparing said range of said measured parameters to known sensory evaluations to determine a sensory quality of pulp in the citrus juice and correlating said sensory quality of pulp to known consumer ratings to determine whether the citrus juice has the type of pulp desired by consumers.

11. The method of claim 10 wherein more than one parameter of said pulp is measured and compared to known sensory evaluations.

12. The method of claim 10 wherein said parameter is selected from the group consisting of length, area, perimeter, fibre width, fibre length, equivalent diameter, and circularity.

13. The method of claim 10 wherein said citrus juice is selected from the group consisting of orange, grapefruit, tangerine, lemon and blends thereof.

14. The method of claim 10 wherein said image based measuring is done using computerized image analysis.

15. The method of claim 10 wherein said parameter measurements and said range of measured parameters are provided to the user in a spread sheet.

16. The method of claim 10 wherein said range of measured parameters is compared to an established relationship between said range of measured parameters and sensory perceived quantity in the mouth to predict a sensory perceived quantity in the mouth for said range of measured parameters.

17. The method of claim 16 wherein said sensory perceived quantity in the mouth for said range of measured parameters is compared to an established relationship between sensory perceived quantity in the mouth and consumer rating of pulp amount to predict a consumer rating for said sensory perceived quantity in the mouth for said range of measured parameters.

18. The method of claim 10 wherein said image based measuring is done using a particle image analyzer.
